(19) United States
(12) Patent Application Publication
(10) Pub. No.: US 2008/0085546 A1
(43) Pub. Date: Apr. 10, 2008

(54) COMPOSITIONS COMPRISING ORGANIC MATTER AND MICROORGANISMS AND METHODS OF USING SAME

(76) Inventors: Eric G. Lancaster, Tucson, AZ (US); Carlis D. King, Alto, TX (US)

Correspondence Address: CONLEY ROSE, P.C.;David A. Rose, P. O. BOX 3267, HOUSTON, TX, 77253-3267, US

(21) Appl. No.: 11/853,410

(22) Filed: Sep. 11, 2007

Related U.S. Application Data
(60) Provisional application No. 60/828,697, filed on Oct. 9, 2006.

(51) Int. Cl. C12N 1/00; C12N 1/12; D06M 16/00

(52) U.S. Cl. 435/252.1; 435/255.1; 435/264

(57) ABSTRACT

A composition comprising an organic component and a biological component wherein the organic component comprises a brix of equal to or greater than about 75%. A composition comprising an organic component, a biological component and water wherein the organic component comprises a brix of equal to or greater than about 75% and the water is present in an amount of from about 80 wt. % to about 99.5 wt. % based on the total weight of the composition. A method comprising contacting equal to or greater than about 3 microorganisms with an organic component in an anaerobic environment under conditions that allow for the fermentation of the composition to produce a fermented composition, and diluting the fermented composition with a diluent to form a diluted fermented composition wherein the ratio of diluent to fermented composition is about 1:50.

### CROSS-REFERENCE TO RELATED APPLICATIONS

The present application claims priority to U.S. Provisional Application Ser. No. 60/828,697 filed Oct. 9, 2006 and entitled “Compositions comprising Organic Matter and Microorganisms and Methods of Using Same” which is incorporated by reference.

### BACKGROUND

This disclosure relates to environmentally safe multipurpose compositions. More specifically, this disclosure relates to compositions comprising an organic material and microorganisms and methods of using same.

Over the past 60 years, a tremendous growth in the use of synthetic chemicals has spread throughout the world. Many of the synthetic chemicals originally intended for commercially regulated use have made their way into the domestic markets where they may be improperly used and discharged into the environment. Within the past decade, a shift toward sustainable technologies has brought to the public's eye the need for “green” cleaners. However, many cleaner formulations continue to contain ammonia, salts, alcohols, and preserving agents which all work on the principals of oxidation.

Attention has recently focused on the role or effect of bacteriocins in controlling the growth of pathogenic bacteria. Bacteriocins are secreted by beneficial microorganisms, namely from lactic acid bacteria, yeasts, and photosynthetic bacteria. Bacteriocins damage the cellular walls of pathogenic bacteria, preventing them from being able to reproduce and eventually killing them without the use of alcohols, oxidizers, or synthetic compounds. The use of bacteriocins to control pathogenic bacteria will not lead to pleomorphs or mutant microorganisms as it is nature's method of controlling growth. Thus, it would be desirable to develop a bacteriocin-based composition and methods of using same.

### SUMMARY

Disclosed herein is a composition comprising an organic component and a biological component wherein the organic component comprises a brix of equal to or greater than about 75%.

Also disclosed herein is a composition comprising an organic component, a biological component and water wherein the organic component comprises a brix of equal to or greater than about 75% and the water is present in an amount of from about 80 wt. % to about 99.5 wt. % based on the total weight of the composition.

Further disclosed herein is a method comprising contacting equal to or greater than about 3 microorganisms with an organic component in an anaerobic environment under conditions that allow for the fermentation of the composition to produce a fermented composition, and diluting the fermented composition with a diluent to form a diluted fermented composition wherein the ratio of diluent to fermented composition is about 1:50.

Further disclosed herein is a method of preparing a composition comprising contacting equal to or greater than 3 microorganisms with an organic component in an anaerobic environment under conditions that allow for the fermentation of the composition to produce a fermented composition, and contacting the fermented composition with a natural soap to form a multipurpose composition.

### DETAILED DESCRIPTION

Disclosed herein are compositions comprising an organic component and a biological component and methods of using same. In an embodiment, the biological component comprises at least three species of microorganisms representing three genus of microorganisms. Each component of the composition will be described in more detail later herein. Such compositions may be multipurpose and for example may be used as a cleaner, deodorizer, degreaser, mold inhibitor, and/or insect repellent. Such compositions may be further characterized as being “environmentally friendly” compositions. Herein “environmentally friendly” is given its ordinary meaning as determined by one of skill in the art and may include compositions that have a minimal impact on the natural environment; that use as well as maintain natural or organic materials; that contain no synthetically derived ingredients; that have a positive impact on nature; that will not cause sickness or malaise; and/or do not produce toxins.

In an embodiment, the composition comprises an organic component. The organic component may be any organic material which functions to support the microbial activity of the microorganisms to be disclosed later herein. Alternatively, the organic component may comprise a carbon source with a brix of equal to or greater than about 75%, alternatively greater than about 77%, alternatively from about 75% to about 90%. A brix is a measurement of the mass ratio of dissolved sucrose to water in a liquid. In an embodiment, the organic component comprises a carbon source with a brix of equal to or greater than about 75% which is present in the composition in an amount of from about 1 wt. % to about 10 wt. %, alternatively from about 3 wt. % to about 5 wt. %, alternatively from about 1 wt. % to about 3 wt. %. The brix of the organic component will decrease upon contacting with the microorganisms of this disclosure as the microorganisms consume the sucrose in the organic component. Examples of carbon sources with a brix of equal to or greater than about 75% include without limitation black strap molasses, citrus molasses and honey.

In an embodiment, the composition comprises a biological component. The biological component may comprise at least three microorganisms, alternatively at least four microorganisms, alternatively at least five microorganisms wherein the microorganisms may be characterized as acid fast, capable of producing lactic acid, producing no butyric acid, being non-pathogenic, and having an antagonistic value of greater than about 50. Herein an antagonistic value of greater than about 50 indicates the microorganisms are capable of growth suppression of 50% or more hyphae of Fusarium cultured in agar media under the conditions of 24 hours of cultivation.

Any microorganism having the aforementioned characteristics may be included in the biological component of the composition. For example, the microorganisms may be selected from the group comprising phototrophic bacteria, lactic acid bacteria, and yeast. In an embodiment, the biological component comprises at least one species of microorganisms selected from each of the members of the group comprising phototrophic bacteria, lactic acid bacteria, and yeast. Alternatively, the biological component comprises more than one species of microorganism selected from each of the members of the group comprising phototrophic bacteria, lactic acid bacteria, and yeast. The final mixture of microorganisms selected for inclusion in the biological component may have a pH of from about 2.7 to about 4.3, alternatively from about 3.0 to about 3.9, alternatively from about 3.3 to about 3.65.

Microorganisms suitable for use in this disclosure comprise phototrophic bacteria such as microorganisms belonging to Rhodopseudomonas, Rhodobacter, Rhodospirillum, Chromatium, and Chlorobium. Specific examples of phototrophic bacteria include Rhodopseudomonas sphaeroides (e.g. IFO 12203), Rhodospirillum rubrum (e.g. IFO 3986), Chromatium okenii and Chlorobium limicola. Lactic acid bacteria include for example and without limitation microorganisms belonging to Lactobacillus, Propionibacterium, Pediococcus, and Streptococcus. Specific examples of lactic acid bacteria include Lactobacillus bulgaricus (e.g. ATCC 11842), Propionibacterium freudenreichii (e.g. IFO 12391), Pediococcus halophilus (e.g. IFO 12172), Streptococcus lactis (e.g. IFO 12007) and Streptococcus faecalis (e.g. IFO 3971). Examples of yeast include without limitation microorganisms belonging to Saccharomyces and Candida. Specific examples of yeast that may be included in these compositions are Saccharomyces cerevisiae (e.g. IFO 0304), Saccharomyces lactis (e.g. IFO 0433) and Candida utilis (e.g. IFO 0396).

In some embodiments, additional microorganisms such as actinomycetes and mold fungi may be included in the compositions of this disclosure. For example, actinomycetes such as microorganisms belonging to Streptomyces, Streptoverticillium, Nocardia, Micromonospora and Rhodococcus genus may be included in the composition. Specific examples of actinomycetes include Streptomyces albus (e.g. ATCC 3004), Streptoverticillium baldaccii (e.g. ATCC 23654), Nocardia asteroides (e.g. ATCC 19247), Micromonospora chalcea (e.g. ATCC 12452) and Rhodococcus rhodochrous (e.g. ATCC 13803). Examples of mold fungi include without limitation microorganisms belonging to Aspergillus and Mucor. Specific examples of these mold fungi include Aspergillus japonicus (e.g. IFO 4060), Aspergillus oryzae (e.g. IFO 4075), and Mucor hiemalis (e.g. IFO 5303).

These microorganisms may be included as their commercial preparations in the biological component of the composition. Alternatively, these microorganisms may be pre-cultured in a proper culture medium before being included in the biological component. The pre-culture may be conducted in a proper culture medium with a pH of from about 3.0 to about 5.0 at a temperature in the range of from about 25° C. to about 45° C. A proper culture medium and conditions for culturing a microorganism depend on the specific microorganism and as such no particular limitation is imposed on the culture medium which is used for this pre-culture. The biological component may be present in the composition in an amount of from about 1% to about 10% based on the total volume of the composition.

In some embodiments, the composition further comprises water such as tap or filtered water. Filtered water may be prepared through any means known to one of ordinary skill in the art; alternatively the water may be filtered through a carbon filter and circulated through a didium boride magnet and far-infrared ceramics. Water may be present in the composition in an amount from about 80% to about 98.0%, alternatively from about 90% to about 98.0% or, alternatively from about 96% to about 98.0% based on the total volume of the composition.

Additives may be included in the composition for a variety of reasons including but not limited to enhancing the aesthetics of the composition, stabilizing the composition, and stimulating the growth of microorganisms in the composition.

The composition may comprise stabilizers that function to promote the stability of the composition such as for example distilled spirits, acetic acid and ceramic powder. In some embodiments, the composition comprises ceramic powder which may be present in the composition in amounts ranging from about 0.001% to about 0.5%, alternatively from about 0.001% to about 0.05%, alternatively from about 0.001% to about 0.02% based on the total weight of the composition.

In at least some embodiments, the composition comprises a distilled spirit. Distilled spirits are well known and widely commercially available and include for example vodka. The distilled spirit may be present in the composition in amounts of from about 1% to about 10% by volume, alternatively from about 3% to about 10%, alternatively from about 1% to about 3%.

In other embodiments, the composition comprises an acid such as for example acetic acid. Acetic acid may be included in the composition in amounts of from about 1% to about 10% by volume, alternatively from about 3% to about 10%, alternatively from about 1% to about 3%.

Yet still further embodiments may comprise organic material such as for example herbs, natural soap, an organic plant-derived foaming agent, an essential oil, spices, and additional phytochemicals including without limitation lavender, sea salt, clays, and inorganic material such as trace minerals. Herein an essential oil refers to any concentrated, hydrophobic liquid containing volatile aroma compound from plants and as is not to be considered a required component of the composition.

In at least some embodiments, the composition is prepared by contacting a biological component and an organic component (both of which have been disclosed herein) in an anaerobic environment at a temperature in the range of from about 50° F. to about 140° F., alternatively from about 65° F. to about 125° F., alternatively from about 95° F. to about 110° F. The biological component and organic component may be contacted using any suitable device and/or container that is compatible with the biological component, organic component, any additives that may be present, and the resulting composition. Without wishing to be limited by theory, the contacting of the biological component and organic component under such conditions may allow for the composition to ferment and such fermentation may be continued until a large percentage of the sugars in the composition are digested and the composition has reached an equilibrium pH of from about 2.7 to about 4.3, alternatively from about 3.0 to about 3.7, alternatively from about 3.3 to about 3.7. The composition may then be aged for equal to or greater than about fifteen days, alternatively equal to or greater than about twenty days, alternatively equal to or greater than about 30 days. During the aging process, any residual sugars may be completely digested and the end product matured. The resulting fermented composition of the biological component and organic component is hereafter referred to as the FC.

In some embodiments, the FC may be used without further processing and will be referred to hereinafter as the concentrated FC. A composition comprising a concentrated FC may serve as one type of multipurpose environmental cleaner (MEC) and hereinafter these compositions are termed MEC1. MEC1 may have a final pH of from about 2.7 to about 3.7, alternatively from about 3.3 to about 3.65, alternatively from about 3.5 to about 3.55.

In some embodiments, the concentrated FC may be contacted with a natural ingredient which provides saponins, such as yucca root or soap bark. This natural ingredient may be added during the fermentation stage. In other embodiments, the concentrated FC may be contacted with a natural soap. Any natural soap may be suitable for use with the concentrated FC disclosed herein. Alternatively, the natural soap may be prepared by the user. The natural soap may be prepared using protocols and techniques known to one of ordinary skill in the art. Alternatively, the natural soap may be prepared by a method comprising contacting a strong base with water to form lye. Suitable strong bases include for example and without limitation sodium hydroxide and potassium hydroxide. The lye may then be heated to a temperature of from about 140° F. to about 180° F. Optionally, oils may be added to the heated lye mixture to perform a variety of functions including for example providing to the final composition an appealing scent. Such oils may also aid in the functions of the MEC such as in the ability of the MEC to deter pests or inhibit mold growth. Such functions will be described in more detail later herein. Oils that may serve such functions would be known to one of ordinary skill in the art. For example, the oils may comprise vegetable oils such as palm oil or coconut oil. The resulting mixture of lye and oil may be cooled and allowed to form a gel. Upon indication of the formation of a gel, a diluent such as for example water may be added to the mixture and the mixture further processed to form a soap. Further processing steps to produce a soap may include additional heating and/or stirring. Additives may be included in the soap to meet a user-desired function or process. For example, such additives may include scents, pigments, dyes and the like. In an embodiment, the soap may be present in the MEC in an amount of from about 0.001% to about 0.05% based on the total volume of the MEC.

In some embodiments, the FC may be contacted with a diluent. For example the diluent may be water. This water may be added in addition to the water used to prepare the FC. The FC may be combined with a diluent at a ratio of FC:diluent of about 1:50, alternatively about 1:20, alternatively about 1:1 and the mixture of diluent and FC is hereinafter referred to as a diluted FC. A composition comprising a diluted FC is hereinafter referred to as MEC2. MEC2 may have a final pH of from about 6 to about 8, alternatively from about 6.5 to about 7.6, alternatively from about 6.9 to about 7.35. In an embodiment, a 100% pure essential oil such as lavender, a stabilizer such as far-infrared ceramic powder, a natural soap or an organic-plant derived foaming agent such as yucca root extract, or liquid soap is added to the MEC2 to both stabilize and improve the fragrance of the MEC2. It is to be understood that the essential oils, stabilizer, natural soap or organic plant derived foaming agents are added to improve the stability and aesthetic qualities of the MEC2. As such these components may be chosen by one of ordinary skill in the art to meet the desired needs of the user. In some embodiments, these additives may also be included in MEC1 where they may serve a similar function.

Hereinafter when referring to properties or characteristics that may apply to both MEC1 and MEC2, the designation MEC will be used. The MEC when prepared as described herein may be used immediately or may be stored for some period of time. In an embodiment, MEC1 may be stored for a period of from about five to about ten years and MEC2 may be stored for a period of from about nine to about twelve months. The MEC may be stored in any container compatible with the components of the MEC in a temperatures range of from 40° F. to about 155° F., alternatively from about 48° F. to about 155° F., alternatively from about 55° to about 85° F. In an embodiment, the MEC may be transferred to a container that has a means for dispensing a predetermined amount of the MEC such as for example and without limitation a spray bottle or a device with a trigger/spray apparatus such as an aerosol can.

In some embodiments, MEC1 may function as a degreaser. MEC1 may function to effect the removal of a wide range of contaminants including oils, greases, sludge bottoms, fats, inks and pigments.

In other embodiments, MEC1 may function as a fungicide. In such embodiments, MEC1 when applied to a material having with at least one type of fungus may effect the killing or growth inhibition of said fungus.

In some embodiments, MEC2 may function as a liquid cleaner. In such embodiments, MEC2 may be contacted with a surface that requires cleaning and MEC2 removed at some later time with an absorbent material such as for example and without limitation a cloth or paper towel. The surface may be wood, glass, plastic, concrete, grout, porcelain, cloth, leather, rubber, metal or combinations thereof and may be further characterized by the presence of dirt, dust, oil, lime scale, grease, stains, salt buildups and the like. In some embodiments, MEC2 may be contacted with a surface having grease, stains, salt buildup or combinations thereof for from about 1 minute to about 4 hours, alternatively for from about 5 minutes to about 1 hour, alternatively for from about 5 to about 10 minutes before being removed with an absorbent material.

In other embodiments, MEC2 may function to support the proper functioning of a sewer system. In such embodiments, MEC2 may be introduced to a sewer system at an inlet port and allowed to remain in the system for some time period before being displaced by another fluid such as for example water. The presence of MEC2 in the sewer system may result in a variety of beneficial outcomes including for example a reduction in the number of pathogenic bacteria in the system and support of the growth of beneficial bacteria in the system.

In other embodiments, MEC2 may function as an insect repellent. Without wishing to be limited by theory, the waste materials generated by the preparation of MEC2 may be utilized for destroying or repelling insects. MEC2 may be applied to an area inhabited by insects such as for example fire ants and following application there may be a reduction in the number of insects inhabiting said area.

In other embodiments, MEC2 may function as a sanitizer which is able to reduce the number of pathogenic microorganisms on a surface to which it is applied. In such embodiments, MEC2 when applied to an area comprising pathogenic bacteria such as for example and without limitation Salmonella results in a reduction in the number of pathogenic bacteria. Furthermore, said area may have an increase in the number of non-pathogenic bacteria such as for example Rhodopseudomonas sphaeroides (e.g. IFO 12203), Lactobacillus bulgaricus (e.g. ATCC 11842), or, Saccharomyces cerevisiae (e.g. IFO 0304). Without wishing to be limited by theory, the bacteriocins present in the MEC may be responsible for controlling the growth of pathogenic bacteria and promoting the growth of nonpathogenic bacteria.

In yet still other embodiments, MEC2 may function as a deodorizer. In such embodiments, MEC2 may be contacted with a surface to improve the odor associated with the surface. Alternatively, MEC2 may be sprayed directly into the air.

In other embodiments, the MEC when prepared as disclosed herein comprises baceteriocins, enzymes, amino acids and trace materials. The MEC may be used for example as a liquid cleaner, degreaser, fungicide, deodorizer, sanitizer, insect repellent or combinations thereof.

### EXAMPLES

The various embodiments having been described, the following examples are given as particular embodiments and to demonstrate the practice and advantages thereof. It is understood that the examples are given by way of illustration and are not intended to limit the specification or the claims in any manner.

### Example 1

A fermented extract, the FC, was prepared as described herein. The FC is a fermented finished product that was aged for a minimum of 30 days and had a final equilibrium pH of 3.1-3.55. It had a light brown color, produced various phenol compounds, had live microbes and an indicator layer of yeast on the surface of the product. The yeast layer was a white film that formed on the surface of the material. The extract was bottled in airtight food grade containers, stored out of direct sunlight and not allowed to freeze. The extract was spoiled if it emitted a foul odor that would be similar to rotten eggs. This extract was used in the following examples.

### Example 2

Filtered water was prepared as follows: tap water was passed through a primary paper filter before being contacted and passed through a secondary carbon filter and finally passed through a tertiary filter Mag-Tec which is a didium boride magnet unit. The water after having been contacted with the tertiary filter was then introduced to a ceramic filter, the 10 35 mm EM-X® Ceramic blaster which had a 1.5″ diameter pipe. Water was allowed to circulate between the tertiary filter and ceramic blaster for a minimum of four passes at a flow rate of 22 gallons per minute.

### Example 3

A MEC was prepared by contacting the FC from Example 1, EM•1® MICROBIAL INNOCULANT commercially available from CEMP USA Inc, fresh herbs, distilled spirit, acetic acid, an organic carbon source and filtered water. 100% pure essential oil of lavender commercially available from Frontier Natural Products Co-op was added to the composition for fragrance. A natural soap, ECOVER dishwashing liquid commercially available from Ecover Belgium N.V. and a far-infrared EM-X® ceramic powder SUPER CERA 7 micron powder commercially available from CEMP USA, Inc was then added to the composition. The final pH of the composition was between 6.9 and 7.35.

### Example 4

Windows were cleaned using conventional cleaners and with the MEC of Example 3. The windows cleaned with the MEC repelled dust for a period twice as long as a conventional ammonia-based window cleaner. The MEC also removed all previous scale and soap film from the windows.

### Example 5

Floors were sprayed until moist with the MEC of Example 3 and wiped clean. Upon drying no spots were visible and all soap films were removed. Finished wood, non-finished wood, tile and laminated floors showed similar results with no detrimental effects.

### Example 6

A car was sprayed until moist with the MEC of Example 3 and wiped clean. Upon drying no spots were visible and all soap films were removed. All surfaces (glass, plastic, chrome, cloth, leather, rubber, tinting on windows, etc.) inside and out were cleaned with no detrimental effects on any of the materials.

### Example 7

Sinks were sprayed until moist with the MEC of Example 3 and wiped clean. Upon drying no spots were visible and all soap films were removed. Porcelain, steel, and plastic sinks were cleaned with no detrimental effects.

### Example 8

Chrome faucets were sprayed until moist with the MEC of Example 3 and wiped clean. Upon drying no spots were visible and all soap films were removed.

### Example 9

Concrete and grout that were stained with grease and oil were treated with the MEC of Example 3 and allowed to soak for a period of 10 minutes. All grease and oil was removed. Motor oil on driveway was treated with the MEC and allowed to soak for 3 hours. 90% of motor oil was visibly removed. A second treatment saturating the area and allowing it to air dry removed the remaining 10% of motor oil.

### Example 10

Fire ants had entered a home and were found colonizing on floors and in cabinets. The MEC of Example 3 was sprayed on the ants. Within 24 hours the ants had moved away from the area sprayed to another location in the home. The process was repeated until the ants had left the home. The final step was reached by spraying the perimeter of the home two separate times within 24-hour intervals. The ants did not return for a period of up to 3 months.

### Example 11

General household odors were eliminated within seconds of spraying the MEC of Example 3 in the air. Items sprayed were garbage cans, litter boxes, bathrooms, and the garage. All areas were free of odors for several hours after being sprayed.

### Example 12

A MEC was prepared using the FC prepared in Example 1 and natural soap. Specifically, to 33 ounces of FC was added 33 ounces of filtered water which were mixed together in a stainless steel pot. 33 ounces of potassium hydroxide was added slowly to the FC and water and heated to a temperature of 180° F. In a separate container 8 ounces of organic coconut oil and 5 ounces of organic palm kernel oil were contacted and heated to a temperature of 180° F. The mixture containing the potassium hydroxide, water and FC was then added to the mixture of oils and the composition was heated and stirred until it began to gel. Upon indication that the mixture started to gel, 10 gallons of water was added to the mixture and the entire composition brought to a boil. The composition was boiled for a minimum of 8 hours and stirred every 10-15 minutes. The resultant soap was then be added to a sample of the FC and the following additives were included; 0.05% Orange Oil; 2% Vodka; 2% Organic Wine Vinegar; 0.5% Organic Raw Honey; 2% Citrus Molasses; 2% EMI MICROBIAL INOCULANT; 0.001% Super C EM-X Ceramic Powder and 2% Magnesium Chloride. The resultant composition was used as a degreaser and to control mold growth. The composition was shown to completely control five major mold groups and destroy mold spores.

While various embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The discussion of a reference herein is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

What is claimed is:

1. A composition comprising an organic component and a biological component wherein the organic component comprises a brix of equal to or greater than about 75%.

2. The composition of claim 1 wherein the organic component is present in an amount of from about 1 wt. % to about 10 wt. % based on the total weight of the composition.

3. The composition of claim 1 wherein the biological component is present in an amount of from about 1% to about 10% based on the total weight of the composition.

4. The composition of claim 1 wherein the biological component comprises a mixture comprising equal to or greater than 3 microorganisms.

5. The composition of claim 4 wherein the microorganisms are acid fast, capable of producing lactic acid, producing no butyric acid, non pathogenic, and have an antagonistic value of greater than about 50.

6. The composition of claim 4 wherein the microorganisms comprise at least one species selected from each member of the group comprising phototrophic bacteria, lactic acid bacteria and yeast.

7. The composition of claim 4 wherein the mixture has a pH of from about 2.7 to about 4.3.

8. The composition of claim 1 further comprising water wherein the water is present in an amount of from about 80 wt. % to about 98.0 wt. % based on the total weight of the composition.

9. The composition of claim 1 further comprising a ceramic powder wherein the ceramic powder is present in an amount of from about 0.001 wt. % to about 0.5 wt. % based on the total weight of the composition.

10. The composition of claim 1 further comprising a distilled spirit wherein the distilled spirit is present in an amount of from about 1% to about 10% based on the total volume of the composition.

11. The composition of claim 1 further comprising an acid wherein the acid is present in an amount of from about 1% to about 10% based on the total volume of the composition.

12. The composition of claim 1 further comprising a soap wherein the soap is present in an amount of from about 0.001% to about 0.05% based on the total volume of the composition.

13. The composition of claim 12 wherein the soap is a natural soap.

14. The composition of claim 1 wherein the composition functions as a cleaner, an insect repellent, a deodorizer, a sanitizer, a degreaser, an anti-mold agent or combinations thereof.

15. A composition comprising an organic component, a biological component and water wherein the organic component comprises a brix of equal to or greater than about 75% and the water is present in an amount of from about 80 wt. % to about 98 wt. % based on the total weight of the composition.

16. A method comprising:
contacting equal to or greater than about 3 microorganisms with an organic component in an anaerobic environment under conditions that allow for the fermentation of the composition to produce a fermented composition; and
diluting the fermented composition with a diluent to form a diluted fermented composition wherein the ratio of diluent to fermented composition is about 1:50.

17. The method of claim 16 wherein the organic component comprises a brix equal to or greater than about 75%.

18. The method of claim 16 wherein the microorganisms comprise at least one species selected from the each member of the group comprising phototrophic bacteria, lactic acid bacteria and yeast.

19. The method of claim 16 wherein the diluent is an aqueous diluent.

20. The method of claim 16 wherein the method further comprises contacting the diluted fermented composition with at least one material selected from the group consisting of: a ceramic powder; a distilled spirit; an acid; and an essential oil.

21. The method of claim 16 wherein the diluted fermented composition is configured to function as at least one selected from the group consisting of: a liquid cleaner; an insect repellent; a deodorizer; and a sanitizer.

22. A method of preparing a composition comprising:
contacting equal to or greater than 3 microorganisms with an organic component in an anaerobic environment under conditions that allow for the fermentation of the composition to produce a fermented composition; and
contacting the fermented composition with a natural soap to form a multipurpose composition.

23. The method of claim 22 wherein the multipurpose composition is configured to function as at least one selected from the group consisting of: a degreaser and an anti-mold agent.
